(19) United States
(12) Patent Application Publication
(10) Pub. No.: US 2005/0002970 A1
(43) Pub. Date: Jan. 6, 2005

(54) Inorganic nanopartices to modify the viscosity and physical properties of ophthalmic and otic compositions

(76) Inventors: Howard Allen Ketelson, Forth Worth, TX (US); David L Meadows, Colleyville, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/494,710

(22) PCT Filed: Dec. 20, 2002
(86) PCT No.: PCT/US02/41249

Related U.S. Application Data
(60) Provisional application No. 60/342,964, filed on Dec. 21, 2001.

(51) Int. Cl. A61K 9/00; A61K 9/14; A61K 33/06

(52) U.S. Cl. 424/400; 424/684; 424/489

(57) ABSTRACT

The use of nanoparticles of inorganic materials (e.g., synthetic smectite clays) in ophthalmic and otic pharmaceutical nanoparticles are utilized to modify the rheological properties of the compositions, so as to enhance the viscosity, flow characteristics, lubricity or other characteristics of the compositions. The invention is particularly directed to the provision of lubricant compositions for the eye and ear, and to enhancement of the viscosity, shear thinning and lubricity of artificial tear compositions.

### BACKGROUND OF THE INVENTION

The present invention is directed to the field of ophthalmic and otic pharmaceutical compositions. The invention is particularly directed to the use of inorganic nanoparticles to enhance the viscosity, shear thinning and other rheological properties of ophthalmic and otic compositions. The invention is also useful with respect to enhancement of the lubricating and wetting properties of ophthalmic compositions, such as artificial tear compositions.

The use of nanoparticles formed from synthetic or natural polymers in ophthalmic compositions has been described in various scientific publications, such as:

Kreuter, J. “Nanoparticles” Colloidal Drug Delivery Systems, edited by Jork Kreuter, Marcel Dekker, New York, N.Y. (USA), chapter 5, page 219 (1994);
Gurny, R. “Ocular therapy with nanoparticles” Polymeric Nano articles and Microspheres edited by P. Guiot and P. Couvreur, Boca Raton, Fla. (USA): CRC Press, page 127 (1986);
Gurny, R. “Preliminary study of prolonged acting drug delivery system for the treatment of glaucoma” Pharm Acta Helv. volume 56, page 130 (1981);
Zimmer, et al. “J. Microspheres and nanoparticles used in ocular delivery systems” Advanced Drug Delivery Reviews, volume 16, number 1, pages 61-73 (1995); and

Calvo, et al. “Comparative in vitro evaluation of several colloidal systems, nanoparticles, nanocapsules, and nanoemulsions, as ocular drug carriers” J Pharm Sci, volume 85, number 5. pages 530-536 (May 1996).

The nanoparticles utilized in the present invention are not formed from synthetic or natural polymers such as those described in the above-cited publications. Rather, the present invention is directed to the use of inorganic nanoparticles. The nanoparticles utilized in the present invention include, for example, clay substances that are water swellable. An extensive review of clays and their chemical and physical properties can be found in:

Giese, R. F. and van Oss C. J., “Colloid and Surface Properties of Clays and Related Minerals”, A. T. Hubbard, Marcel Dekker Inc., Vol. 105.

The preferred nanoparticles are formed from synthetic smectite clays which are prepared from simple silicates. The following publications may be referred to for further background regarding the use of synthetic clay nanoparticles in pharmaceutical compositions:
Plaizier-Vercammen, “Rheological properties of Laponite XLG, a synthetic purified hectorite” Pharmazie, volume 47, page 856 (1992);
Grandolini, et al. “Intercalation compounds of hydrotalcite-like anionic clays with anti-inflammatory agents: I. Intercalation and in vitro release of ibuprofen” International Journal of Pharmaceutics, volume 220, numbers 1-2, pages 23-32 (Jun. 4, 2001);
U.S. Pat. No. 5,585,108 (Ruddy, et al.) entitled “Formulations of Oral Gastrointestinal Therapeutic Agents in Combination with Pharmaceutically Acceptable Clays”;
U.S. Pat. No. 6,177,480 B1 (Tsuzuki, et al.), which describes the use of a synthetic clay material (i.e., Laponite™) as a wetting agent for contact lenses and to assist in the removal of lipid deposits from contact lenses by surfactants;
U.S. Pat. No. 6,015,816 (Kostyniak, et al.), which describes an improved method using colloid particles, such as smectite clay minerals, as a substrate for ligands having antimicrobial activity, so as to control microbial growth on a material; and

U.S. Pat. No. 6,177,480 (Tsuzuki, et al.) describes the use of synthetic clay material (i.e., Laponite™) as a wetting agent for contact lenses and to assist in the removal of lipid deposits from contact lenses by surfactants.

For a recent review of rheology modifiers available for use in various applications, see: Braun, et al., “Practical use & application” Rheology Modifiers Handbook, William Andrews Publishing, New York, N.Y. (USA) (2000).

The use of inorganic nanoparticles of the type described herein to modify the physical properties of ophthalmic and otic pharmaceutical compositions has not been disclosed or suggested in the prior art.

### SUMMARY OF THE INVENTION

The present invention is based on the use of inorganic nanoparticle materials to facilitate the formulation of ophthalmic and otic compositions, particularly compositions adapted for topical application to ophthalmic or otic tissues. The use of synthetic inorganic nanoparticles is preferred. The inorganic nanoparticles described herein are particularly well suited for use in ophthalmic and otic pharmaceutical compositions wherein control of the rheological properties of the compositions is needed. The nanoparticles may be utilized for this purpose, either alone or in combination with well-known rheological additives, such as cellulosics, acrylic polymers, guars, carrageenans, alginates, xanthan gums, and polyvinyl pyrrolidone polymers.

The present invention is particularly directed to the use of inorganic nanoparticles to modify the viscosity, shear thinning and other Theological properties of artificial tears and ocular lubricants, so as to simulate the physical properties of mucin in normal tear fluids. The invention is also directed to improving the comfort of contact lens wearers and dry eye patients by enhancing the lubricating and wetting properties of ophthalmic compositions.

It has been shown that mucin in tears plays a major physical function in producing shear-thinning behavior. Model solutions containing mucin have been shown to have viscosity-shear rate curves which are similar to human tears (see the work reported by Tiffaizy, et. al, in Lacrimal Gland, Tear Film, and Dry Eve Syndromes 2, page 229, (Sullivan, et al., editors; Plenium Press, NY, 1998). Viscosity shear rate curves showed that both unstimulated and stimulated human tears have viscosities that decrease from approximately 9 mP*sec at very low shear rates (e.g., less than 0.2 sec⁻¹) to a newtonian plateau viscosity of approximately 1.0 at higher shear rates (e.g., greater than 10 sec⁻1). One of the objectives of the present invention is to provide ophthalmic compositions that duplicate or simulate these properties.

The present invention is based in part on a finding that aqueous dispersions of the inorganic nanoparticles described herein have shear thinning properties that are quite useful in connection with ocular or otic lubricant products, particularly artificial tear formulations and formulations utilized during ocular surgical procedures. An example of the latter type of formulation is a lubricating, shear thinning formulation utilized to facilitate the formation of a corneal flap with a microkeratome, in conjunction with LASIK surgery.

### BRIEF DESCRIPTION OF THE DRAWINGS

FIG. 1 is a graph showing the results of shear thinning measurements described in Example 2; and

FIG. 2 is a graph showing the results of the shear thinning measurements described in Example 3.

### DETAILED DESCRIPTION OF THE INVENTION

The nanoparticles utilized in the present invention are inorganic materials. The particles have colloidal dimensions, a large surface area and a high ion exchange capacity. The particles are generally referred to hereinafter as “synthetic inorganic nanoparticles”.

The inorganic nanoparticles used in the present invention preferably have particle dimensions less than 100 nanometers (“nm”), but greater than 1 nm. The morphology of the nanoparticles is not limited to being spherical; plate-like, cubic, ellipsoid or other particle shapes are also useful. The particles have surface areas ranging from 30-1000 square meters/gram (“m²/g”), and have an overall negative surface charge at a pH in the range of 6.0 to 7.8.

The inorganic nanoparticles utilized in the present invention may also be surface modified, depending on the particular type of composition involved and stability requirements. Different types of nanoparticles may be combined to optimize the formulation properties.

The inorganic nanoparticles utilized in the present invention are preferably formed from clays that swell in aqueous solutions. These types of clays are referred to herein as being “hydrous”. The use of nanoparticles of synthetic hydrous clays is preferred due to the commercial availability, purity, and well-defined chemical composition and physical properties of these materials. In addition, the synthetic clay nanoparticles are easier to formulate and can form colorless and transparent gels more readily than inorganic nanoparticles formed from naturally occurring clays.

Synthetic inorganic nanoparticles that are particularly useful include a synthetic smectite clay that is commercially available under the trademark Laponite® (Southern Clay Products, Gonzales, Tex., USA). Laponite® is a layered hydrous magnesium silicate prepared from simple silicates. The following publication may be referred to for further details concerning the physical properties and functions of Laponite®: “Laponite Technical Bulletin “Laponite-synthetic layered silicate—its chemistry, structure and relationship to natural clays” L204/01g. Another synthetic magnesium aluminum silicate material is also commercially available under the trademark OPTIGEL® SH (Sud-Chemie, Louisville, Ky.).

Inorganic nanoparticles formed from naturally occurring hydrous clays may also be utilized, either in combination with a synthetic clay or alone. Examples of suitable naturally occurring clays include aliettite, beidellite, bentonite, hectorite, kaolinite, magadite, montinorillonite, nontronite, saponite, sauconite, stevensite and volkonskoite.

The following publications may be referred to for further details regarding the physical properties of various types of clay nanoparticles and the use of these materials as ion-exchange materials, viscosity modifiers and film forming agents:
Gieseking, J. E., “Mechanism of Cation Exchange in the Mont-Morillonite-Beidellite-Nontronite Type of Clay Minerals”, Soil Science, volume 47, pages 1-14 (1939);
Theng, B. K. G., “Formation and Properties of Clay-Polymer Complexes”, Elsevier, Amsterdam, (1979); and
H. van Olphen, “Clay Colloid Chemistry”, Krieger Publishing Company, Florida, Second Edition (1991).

Examples of other inorganic nanoparticle materials that may be utilized instead of or in combination with the clay nanoparticles described above include zeolites, silica, aluminum oxide, cerium oxide, titanium oxide and zinc oxide. Nanometer sized silica particles, such as those supplied by Nalco (e.g., Nalco® 115 and 1140) and EKA Chemicals (NYACOL® grades), are readily available. Mineral oxide nanoparticles based on other metals are also commercially available. For example, mineral oxides (e.g., aluminum oxide, cerium oxide, titanium oxide and zinc oxide) having well defined nano-dimensions are available from Nanophase Technologies (Romeoville, Ill., USA) under the trade name “NanoTek®”.

The incorporation of inorganic nanoparticles in aqueous ophthalmic and otic compositions as described herein results in significant viscosity changes. The compositions of the present invention will typically have viscosities that are orders of magnitude higher than the viscosities of compositions that are identical, except for the inclusion of synthetic inorganic nanoparticles. The compositions of the present invention will preferably have a viscosity of less than 5.0 millipascal second (“mPa*sec”) at high shear rates. More specifically, the compositions of the present invention preferably have Newtonian plateau viscosities of less than 5 mPa*sec at shear rates above 25 sec⁻¹, with viscosities in the range of 0.1 to 1 mPa*sec being most preferred.

The concentration of the inorganic nanoparticles utilized in specific ophthalmic or otic compositions of the present invention will depend on the physical form of the composition (e.g., solution, dispersion, suspension or gel) and other factors apparent to those skilled in the art. The identification of an ideal concentration of nanoparticles for a specific formulation can be determined by means of routine experimentation, conducted in accordance with the specifications and considerations described herein. The ideal concentrations selected as a result of such testing may vary significantly from formulation to formulation, but the concentrations will generally fall within the range of 0.1 to 10 w/v %. The concentration of dispersed smectite clay nanoparticles (e.g., Laponite®) in the compositions of the present invention may vary significantly from formulation to formulation, but is normally within the range of 0.1 to 1 w/v %, and preferably within the range of 0.3 to 0.5 w/v %.

It has been found that at low concentrations in aqueous buffered solutions, the above-described inorganic nanoparticles can be dispersed under physiological pH conditions while retaining a transparent solution, dispersion or gel. The inorganic nanoparticles will form clear and colorless dispersions of low viscosity at concentrations of up to 10 w/v %. However, if combined with appropriate amounts of salts and other excipients, the nanoparticles will form clear, highly shear thing, thixotropic gels. More particularly, at concentrations of greater than 0.5 weight/volume percent (“w/v %”), the particles will form clear gels under appropriate electrolyte conditions and display lubrication, film forming and viscoelastic properties.

The electrolyte conditions required for the formation of such gels will vary somewhat depending on the particular type of inorganic nanoparticle selected, the concentration utilized, the type of buffer or vehicle involved and other factors apparent to persons skilled in the art. However, the preferred electrolyte conditions will generally involve the use of very low levels of 1:1 electrolytes (e.g., NaCl). The ideal concentration of the electrolyte in the gel compositions of the present invention can be readily determined through routine experimentation for each formulation. However, the amount of electrolyte required will generally be on the order of 0.01 to 0.1 w/v %.

The ophthalmic and otic compositions of the present invention may contain various substances in addition to the above-described synthetic inorganic nanoparticles, such as surfactants, buffers and viscosity adjusting agents. The ophthalmic and otic compositions of the present invention will generally be formulated as sterile aqueous solutions, suspensions, dispersions or gels. The compositions must be formulated so as to be compatible with ophthalmic and otic tissues. The ophthalmic solutions, suspensions and dispersions of the present invention will generally have an osmolality of from about 200 to about 400 milliosmoles/kilogram water (“mOsm/kg”). All of the compositions of the invention will have a physiologically compatible pH.

The inorganic nanoparticles described above may be utilized to modify the viscosity, shear thinning and other rheological properties of various types of ophthalmic and otic compositions, including solutions, suspensions, ointments and gels. However, the invention is particularly directed to modification of the physical properties of artificial tear solutions and other types of ophthalmic solutions upon topical application to the eye.

As indicated above, the present invention is particularly useful for modifying the rheological properties of ophthalmic compositions that function as artificial tears or ocular lubricants. Such compositions may contain one or more electrolytes or other substances to simulate the chemical composition of human tears, as described in U.S. Pat. No. 5,403,598 (Beck, et al.). The compositions may also contain one or more polymers, such as carboxy vinyl polymers or galactomannans (e.g., guar and hydroxypropyl guar). The use of galactomannan polymers in such compositions is described in U.S. Pat. No. 6,403,609 (Asgharian); the entire contents of the foregoing patent are hereby incorporated in the present specification by reference.

The present invention may also be employed to modify the viscosity and/or other rheological properties of various types of ophthalmic and otic compositions that contain therapeutically active substances. The compositions of the present invention may therefore contain various types of pharmaceutically active agents, such as agents for controlling intraocular pressure and treating glaucoma, neuroprotectants, anti-allergy agents, anti-infectives, anti-inflammatory agents, mucosecretagogues, angiostatic steroids, pain relievers, demulcents, decongestants or astringents, and so on.

Examples of pharmaceutically active agents which may be included in the compositions of the present invention, and administered via the methods of the present invention include, but are not limited to: anti-glaucoma agents, such as apraclonidine, brimonidine, betaxolol, timolol, pilocarpine, carbonic anhydrase inhibitors and prostaglandins; dopaminergic antagonists; anti-infectives, such as moxifloxacin, gatifloxacin, ciprofloxacin and tobramycin; non-steroidal and steroidal anti-inflammatories, such as rimexolone, dexamethasone, prednisolone, fluorometholone, lotoprednol, naproxen, diclofenac, suprofen, and ketorolac; proteins; and growth factors, such as epidermal growth factor; mucosecretagogues, such as 15-HETE; angiostatic steroids, such as anecortave acetate; antihistamines, such as emadine; mast cell stabilizers, such as olopatadine; and demulcents, such as hydroxypropyl methyl cellulose (“HPMC”), propylene glycol and glycerin.

The ophthalmic and otic compositions of the present invention that are packaged as multi-dose products may contain one or more ophthalmically acceptable biocides in an amount effective to prevent microbial contamination of the compositions by microbes, such as bacteria and fungi. The biocides utilized for this purpose are referred to herein as “antimicrobial preservatives”.

The invention is not limited relative to the types of biocides that may be utilized as antimicrobial preservatives. The preferred biocides include: chlorhexidine, polyhexamethylene biguanide polymers (“PHMB”), polyquaternium-1, and the amino biguanides described in co-pending U.S. patent application Ser. No. 9/581,952 and corresponding International (PCT) Publication No. WO 99/32158, the entire contents of which are hereby incorporated in the present specification by reference. The use of surface-active biocides is preferred.

The preferred antimicrobial agents are polyquaternium-1 and amino biguanides of the type described in U.S. patent application Ser. No. 09/581,952 and corresponding International (PCT) Publication No. WO 99/32158. The most preferred amino biguanide is identified in U.S. patent application Ser. No. 09/581,952 and corresponding PCT publication as “Compound Number 1”, and has the following structure: 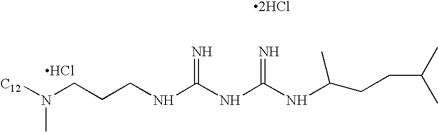
This compound is referred to below by means of the code number “AL8496”.

The levels of antimicrobial activity required to preserve ophthalmic and otic pharmaceutical compositions from microbial contamination are well known to those skilled in the art, based both on personal experience and official, published standards, such as those set forth in the United States Pharmacopoeia (“USP”) and similar publications in other countries. The amount of antimicrobial preservative required for this purpose is referred to herein as “an effective amount”.

The compositions may also contain one or more components to enhance the antimicrobial activity of the compositions, such as: a borate/polyol complex (e.g., boric acid/propylene glycol), as described in U.S. Pat. No. 6,143,799 (Chowhan, et al.); a low molecular weight amino alcohol (e.g., AMP), as described in U.S. Pat. No. 6,319,464 B2 (Asgharian); or a low molecular weight amino acid (e.g., glycine), as described in U.S. Pat. No. 5,741,817 (Chowhan, et al.). The entire contents of the above-referenced patents are hereby incorporated in the present specification by reference. The above-cited components may be used either alone or in combination with conventional antimicrobial agents such as polyquaternium-1.

### EXAMPLE 1

The preferred compositions of the present invention are further illustrated by the formulations described in the following table, which contain synthetic inorganic smectite clay nanoparticles (i.e., Laponite® XLG). All of the concentrations shown in the table are expressed as weight/volume percent.

| Ingredient | 9534-36A | 9534-36B | 9534-36C | 9534-36D | 9534-36E |
|---|---|---|---|---|---|
| Laponite ® XLG | 0.1 | 0.1 | 0.1 | 0.25 | 0.25 |
| Poloxamine | 0.1 | 0.1 | 0.1 | 0.1 | 0.1 |
| 1304 | | | | | |
| Sodium Chloride | 0.5 | — | 0.5 | 0.5 | — |
| Potassium | 0.05 | — | 0.05 | 0.05 | — |
| Chloride | | | | | |
| HPMC | 0.3 | — | — | 0.3 | — |
| Sodium Borate | 0.35 | 0.35 | 0.35 | 0.35 | 0.35 |
| Purified Water | QS | QS | QS | QS | QS |
| pH | 7.8 | 7.8 | 7.8 | 7.8 | 7.8 |
| *Viscosity mPa * s | Newtonian | Newtonian | Newtonian | Newtonian | Newtonian |
| determined at | Behavior | Behavior | Behavior | Behavior | Behavior |
| a shear rate of | 7.19 ± 0.10 | 0.91 ± 0.01 | 1.09 ± 0.01 | 9.43 ± 0.01 | 1.40 ± 0.01 |
| 85.61s⁻¹ | | | | | |

*Determined using Brookfield DVIII+ with a ULA spindle-room temperature at 23° C.

The formulations described in the foregoing table were prepared and evaluated using the following procedures and experimental set-up. In a 600 ml beaker was added 400 ml of purified water. A mixer (Heildolph RZR 2041) was fitted with a 3-bladed stainless steel propeller stirrer and used to mix the formulation. The beaker containing the water was placed on a hot plate and mixed at 200 rpm using the mixer. When the temperature of the water reached 85° C., the appropriate amount of Laponite was added and the dispersion was mixed at 600 rpm for an additional 30 minutes. The heat was subsequently removed and, while still mixing, the dispersion was allowed to equilibrate to room temperature. In another 100 mL beaker, the remaining formulation components were added and dissolved in 80 ml of purified water. The resulting solution was slowly added to the Laponite dispersion while it was mixed at 600 rpm. The pH was adjusted using HCl(aq) and NaOH (aq). Purified water was added to make up the final volume to 100% batch.

The viscosity profiles of the samples were measured using a Brookfield DVIII+ rheometer interfaced to a computer. The rheometer was controlled using the Rheocalc V2.2 software. For each run, approximately 13 ml of sample were added to a ULA-35YZ sample tube fitted in a ULA-40Y water jacket that was equilibrated to 23° C. using a water bath. A YULA-15Z spindle was used for all measurements. The shear rate parameters were pre-set using the Rheocalc software.

### Example 2

The compositions of the present invention are illustrated by the formulations described in the following table, wherein all concentrations are expressed as weight/volume percent.

Formulation Number

| Ingredient | 9819-35A | 9819-35B | 9819-35D |
|---|---|---|---|
| Laponite XLG | 0.3 | 0.3125 | 0.35 |
| Lot 00/211 | | | |
| Propylene Glycol | 1.2 | 1.2 | 1.2 |
| Boric Acid | 0.6 | 0.6 | 0.6 |
| Purified Water | QS | QS | QS |
| pH | 7.8 | 7.8 | 7.8 |

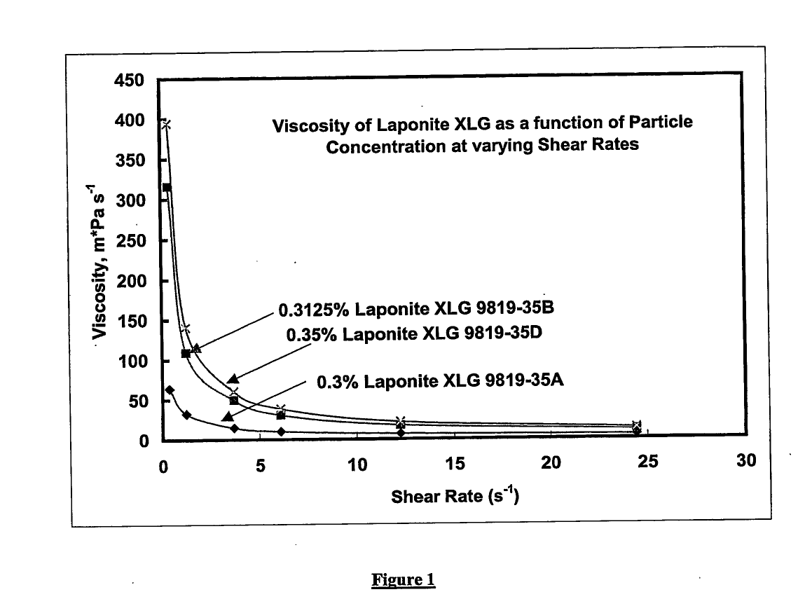

The shear thinning properties of the formulations described above were evaluated by means of the procedures described in Example 1. The results are shown in FIG. 1. The results demonstrate that with the formulations using propylene glycol and boric acid, nanoparticle concentrations of greater than 0.3% provided significant shear thinning properties to the formulation at shear rates between 0.1 s⁻¹ and 5.0 s⁻¹.

### Example 3

The compositions of the present invention were also evaluated over time by monitoring the shear thinning properties of the formulations. The compositions evaluated are shown in the table below, wherein all amounts are expressed as weight/volume percent.

Formulation Number

| Ingredient | 9819-69A | 9819-69B |
|---|---|---|
| Laponite XLG | 0.6 | 0.4 |
| (Lot 00/211) | | |
| Propylene Glycol | 1.2 | 1.2 |
| Boric Acid | 0.4 | 0.4 |
| Purified Water | QS | QS |
| pH | 7.8 | 7.8 |

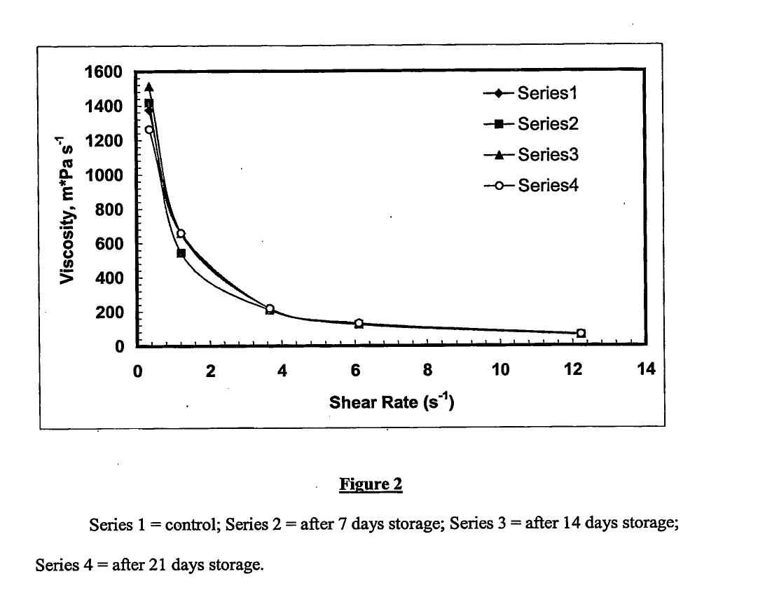

The shear thinning properties of the formulations were evaluated over a three-week period at room temperature, using the procedures described in Example 1. As shown in FIG. 2, there were no significant changes in shear thinning properties.

1. Use of inorganic nanoparticles to modify the viscosity or other physical properties of an ophthalmic or otic pharmaceutical composition.

2. A method of lubricating an eye or ear, which comprises applying an ophthalmic composition containing a lubricating effective amount of inorganic nanoparticles to the eye or ear

3. An ophthalmic or otic pharmaceutical composition, comprising an amount of inorganic nanoparticles sufficient to modify the viscosity or other physical properties of the composition.

4. A composition according to claim 3 wherein the nanoparticles have: (a) particle dimensions less than 100 nm, but greater than 1 nm; (b) surface areas ranging from 30 to 1000 m²/g; and (c) an overall negative surface charge at a pH in the range of 6.0 to 7.8.

5. A composition according to claim 3, wherein the composition has a viscosity of 0.1 mPa*s to 10,000 mPa*s at a shear rate of 20 s⁻¹ to 0.01 s⁻¹.

6. A composition according to claims 3, 4 or 5, wherein the nanoparticles are formed from a synthetic clay material.

7. A composition according to claim 6, wherein the synthetic clay material comprises a smectite clay.

8. A composition according to claims 3, 4 or 5, wherein the nanoparticles are selected from the group consisting of zeolites, hydrotalcite, silica, aluminum oxide, cerium oxide, titanium oxide and zinc oxide.

9. A composition according to claims 3, 4 or 5, wherein the composition is a solution.

10. A composition according to claims 3, 4 or 5, wherein the composition is a thixotropic gel.
